(19) United States
(12) Patent Application Publication
(10) Pub. No.: US 2004/0022727 A1
(43) Pub. Date: Feb. 5, 2004

(54) Aptamer-toxin molecules and methods for using same

(76) Inventors: Martin Stanton, Stow, MA (US); Markus Kurz, Newton, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/600,007

(22) Filed: Jun. 18, 2003

Related U.S. Application Data
(60) Provisional application No. 60/390,042, filed on Jun. 18, 2002.

(51) Int. Cl. A61K 51/00; A61K 39/395; C07K 16/46

(52) U.S. Cl. 424/1.49; 424/178.1; 530/391.1

(57) ABSTRACT

Materials and methods are provided to prepare therapeutic conjugates for the treatment of proliferative diseases. The therapeutic conjugates of the invention comprise a targeting moiety conjugated to a therapeutic moiety. The therapeutic moiety of the conjugates of the present invention have a cytotoxic effect and are useful in the treatment of proliferative diseases.

### REFERENCE TO RELATED APPLICATIONS

This application claims priority to and is related to U.S. Provisional Application Ser. No. 60/390,042, filed Jun. 18, 2002 and is incorporated by reference herein.

### FIELD OF THE INVENTION

The invention relates generally to the field of nucleic acids and more particularly to compositions and methods for delivering cytotoxic agents to cells by linking a nucleic acid aptamer to cytotoxic agents and delivering the aptamer-toxin conjugate to a target. Similarly, a nucleic acid sensor molecule (NASM) can be linked to a toxin and the NASM-toxin conjugate delivered to a target.

### BACKGROUND OF THE INVENTION

Aptamers are nucleic acid molecules having specific binding affinity to non-nucleic acid or nucleic acid molecules through interactions other than classic Watson-Crick base pairing. Aptamers are described e.g., in U.S. Pat. Nos. 5,475,096; 5,270,163; 5,589,332; 5,589,332; and 5,741,679, each of which is incorporated in its entirety by reference herein.

Aptamers, like peptides generated by phage display or monoclonal antibodies (MAbs), are capable of specifically binding to selected targets and, through binding, blocking their targets' ability to function. Created by an in vitro selection process from pools of random sequence oligonucleotides (FIG. 1), aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarity, hydrophobic contacts, steric exclusion, etc.) that drive affinity and specificity in antibody-antigen complexes.

Aptamers have a number of desirable characteristics for use as therapeutics including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial therapeutic leads. In vitro selection allows the specificity and affinity of the aptamer to be tightly controlled and allows the generation of leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC and the immune response is generally trained not to recognize nucleic acid fragments).

3) Administration. Whereas all currently approved antibody therapeutics are administered by intravenous infusion (typically over 2-4 hours), aptamers can be administered by subcutaneous injection. This difference is primarily due to the comparatively low solubility and thus large volumes necessary for most therapeutic MAbs. With good solubility (>150 mg/ml) and comparatively low molecular weight (aptamer: 10-50 KD; antibody: 150 KD), a weekly dose of aptamer may be delivered by injection in a volume of less than 0.5 ml. Aptamer bioavailability via subcutaneous administration is >80% in monkey studies (Tucker, 1999).

4) Scalability and cost. Therapeutic aptamers are chemically synthesized and consequently can be readily scaled as needed to meet production demand. Whereas difficulties in scaling production are currently limiting the availability of some biologics (e.g., Enbrel, Remicade) and the capital cost of a large-scale protein production plant is enormous (e.g., $500 MM, Immunex), a single large-scale synthesizer can produce upwards of 100 kg oligonucleotide per year and requires a relatively modest initial investment (e.g., <$10 MM, Avecia). The current cost of goods for aptamer synthesis at the kilogram scale is estimated at $500/g, comparable to that for highly optimized antibodies. Continuing improvements in process development are expected to lower the cost of goods to <$100/g in five years.

5) Stability. Therapeutic aptamers are chemically robust. They are intrinsically adapted to regain activity following exposure to heat, denaturants, etc. and can be stored for extended periods (>1 yr) at room temperature as lyophilized powders. In contrast, antibodies must be stored refrigerated.

Cytotoxic agents are molecules that have lethal or growth inhibiting effects on cells. Cytotoxic or chemotherapeutics agents can be classified as tubulin stabilizers or destabilizers, anti-metabolites, purine synthesis inhibitors, nucleoside analogs, and DNA alkylating or other DNA modifying agents. Such agents have been used as therapeutics in proliferative diseases such as cancer, solid tumors, inflammation diseases, overactive scarring disorders, and autoimmune diseases such as lupus. Because of their cytotoxic effect these chemotherapeutic agents tend to also affect or inhibit healthy or non-target cells leading to undesirable morbidity or side effects in subjects or patients being treated.

There is a need for delivery of cytotoxic or therapeutic agents to treat proliferative diseases that maximize cytotoxity to diseased malignant cells or target cells without collateral cytotoxicity to healthy or normal cells or surrounding tissue.

The materials and methods of the present invention provide a target specific therapeutic agent-aptamer complex that increases the effectiveness of cytotoxic agents or therapeutics and minimizes damage to non-target cells. The aptamer-toxin conjugates and methods of the present invention meet these and other needs.

### BRIEF DESCRIPTION OF THE DRAWINGS

FIG. 1 shows the in vitro aptamer selection (SELEX™) process from pools of random sequence oligonucleotides.

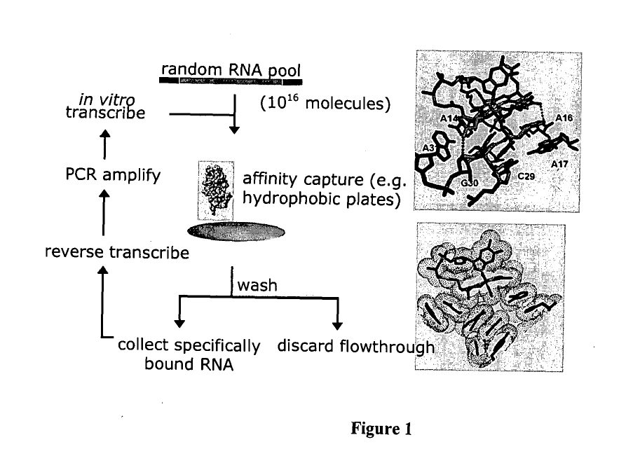

FIG. 2 shows a schematic diagram in which the oligonucleotide population is screened for a nucleic acid sensor molecule which comprises a target molecule activatable ligase activity.

FIG. 3 shows the hammerhead nucleic acid sensor molecule selection methodology.

### SUMMARY OF THE INVENTION

The specificity of aptamers allows them to be used as molecular “chaperones” to increase the specificity of another molecule to a given target by linking said molecule to an aptamer with high binding affinity to a target.

In one embodiment, a cytotoxic agent or toxin is linked to an aptamer, forming a toxin-aptamer conjugate molecule that increases the specificity of the cytotoxic agent moiety to a given specific target. In one embodiment of the toxin-aptamer conjugate, the toxin or cytotoxic agent is a chemotoxin.

In one embodiment, the aptamer-toxin conjugate is used as a chemotherapeutic agent in the treatment of proliferative diseases including, but not limited to, inflammation disorders, scarring, solid tumor cancers, autoimmune disorders, including lupus for instance.

In another embodiment, the toxin conjugate is a protein toxin. In one embodiment, the protein is an antibody or antibody fraction. In another embodiment the toxin is a protein having binding specificity and affinity for another molecule.

In another embodiment, the toxin is a nucleic acid toxin.

In another embodiment, the chemotoxin conjugate is a small molecule therapeutic agent including but not limited to tubulin stabilizers/destabilizers, anti-metabolites, purine synthesis inhibitors, nucleoside analogs, and DNA alkylating or other DNA-modifying agents, including for instance doxorubicin.

In another embodiment, the chemotoxin conjugate includes but is not limited to calichomycin, doxorubicin, taxol, methotrexate, gencitadine, AraC (cytarabine), vinblastin, daunorubicin.

In another embodiment, the toxic agent is a radioisotope.

In another embodiment, the targets for the toxin-aptamer conjugate are cell surface receptors, including but not limited to receptor tyrosine kinases, EGFR, her2 new, PSMA, and Muc1.

The specificity of NASMs allows them to be used as molecular “chaperones” to increase the specificity of another molecule to a given target by linking said molecule to a NASM which recognizes a target with high specificity.

In one embodiment, a cytotoxic agent or toxin is linked to a NASM, forming a toxin-NASM conjugate molecule that increases the specificity of the cytotoxic agent moiety to a given specific target. In one embodiment of the toxin-NASM conjugate, the toxin or cytotoxic agent is a chemotoxin.

In one embodiment, the NASM-toxin conjugate is used as a chemotherapeutic agent in the treatment of proliferative diseases including, but not limited to, inflammation disorders, scarring, solid tumor cancers, autoimmune disorders, including lupus for instance.

In another embodiment, the toxin conjugate is a protein toxin. In one embodiment, the protein is an antibody or antibody fraction. In another embodiment the toxin is a protein having binding specificity and affinity for another molecule.

In another embodiment, the toxin is a nucleic acid toxin.

In another embodiment, the chemotoxin conjugate is a small molecule therapeutic agent including but not limited to tubulin stabilizers/destabilizers, anti-metabolites, purine synthesis inhibitors, nucleoside analogs, and DNA alkylating or other DNA-modifying agents, including for instance doxorubicin.

In another embodiment, the chemotoxin conjugate includes but is not limited to calichomycin, doxorubicin, taxol, methotrexate, gencitadine, AraC (cytarabine), vinblastin, daunorubicin.

In another embodiment, the toxic agent is a radioisotope.

In another embodiment, the targets for the toxin-NASMs conjugate are cell surface receptors, including but not limited to receptor tyrosine kinases, EGFR, her2 new, PSMA, and Muc1.

### DETAILED DESCRIPTION OF THE INVENTION

Definitions

As defined herein, “toxin” is a molecule having a deleterious effect on another molecule or living cell, potentially resulting in the ultimate death of the cell.

As defined herein, “nucleic acid” means either DNA, RNA, single-stranded or double-stranded, and any chemical modifications thereof Modifications include, but are not limited to, those which provide other chemical groups that incorporate additional charge, polarizability, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

As defined herein, “oligonucleotide” is used interchangeably with the term “nucleic acid” and includes RNA or DNA (or RNA/DNA) sequences of more than one nucleotide in either single strand or double-stranded form. A “modified oligonucleotide” includes at least one nucleotide residue with any of: an altered internucleotide linkage(s), altered sugar(s), altered base(s), or combinations thereof.

As defined herein, “target” means any compound or molecule of interest for which a nucleic acid ligand exists or can be generated. A target molecule can be naturally occurring or artificially created, including a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc. without limitation.

As defined herein, a nucleic acid sensor molecule which “recognizes a target molecule” is a nucleic acid molecule whose activity is modulated upon binding of a target molecule to the target modulation domain to a greater extent than it is by the binding of any non-target molecule or in the absence of the target molecule. The recognition event between the nucleic acid sensor molecule and the target molecule need not be permanent during the time in which the resulting allosteric modulation occurs. Thus, the recognition event can be transient with respect to the ensuing allosteric modulation (e.g., conformational change) of the nucleic acid sensor molecule.

As defined herein, a molecule which “naturally binds to DNA or RNA” is one which is found within a cell in an organism found in nature.

As defined herein, a “random sequence” or a “randomized sequence” is a segment of a nucleic acid having one or more regions of fully or partially random sequences. A fully random sequence is a sequence in which there is an approximately equal probability of each base (A, T, C, and G) being present at each position in the sequence. In a partially random sequence, instead of a 25% chance that an A, T, C, or G base is present at each position, there are unequal probabilities.

As defined herein, an “aptamer” is a nucleic acid which binds to a non-nucleic acid target molecule or a nucleic acid target through non-Watson-Crick base pairing.

As defined herein, an aptamer nucleic acid molecule which “recognizes a target molecule” is a nucleic acid molecule which specifically binds to a target molecule.

As defined herein, a “nucleic acid sensor molecule” or “NASM” refers to either or both of a catalytic nucleic acid sensor molecule and an optical nucleic acid sensor molecule.

As defined herein, a “nucleic acid ligand” refers to either or both an aptamer or a NASM.

As defined herein, a “catalytic nucleic acid sensor molecule” is a nucleic acid sensor molecule comprising a target modulation domain, a linker region, and a catalytic domain.

As defined herein, an “optical nucleic acid sensor molecule” is a catalytic nucleic acid sensor molecule wherein the catalytic domain has been modified to emit an optical signal as a result of and/or in lieu of catalysis by the inclusion of an optical signal generating unit.

As defined herein, a “target modulation domain” (TMD) is the portion of a nucleic acid sensor molecule which recognizes a target molecule. The target modulation domain is also sometimes referred to herein as the “target activation site” or “effector modulation domain”.

As defined herein, a “catalytic domain” is the portion of a nucleic acid sensor molecule possessing catalytic activity which is modulated in response to binding of a target molecule to the target modulation domain.

As defined herein, a “linker region” or “linker domain” is the portion of a nucleic acid sensor molecule by or at which the “target modulation domain” and “catalytic domain” are joined. Linker regions include, but are not limited to, oligonucleotides of varying length, base pairing phosphodiester, phosphothiolate, and other covalent bonds, chemical moieties (e.g., PEG), PNA, formacetal, bismaleimide, disulfide, and other bifunctional linker reagents. The linker domain is also sometimes referred to herein as a “connector” or “stem”.

As defined herein, an “optical signal generating unit” is a portion of a nucleic acid sensor molecule comprising one or more nucleic acid sequences and/or non-nucleic acid molecular entities, which change optical or electrochemical properties or which change the optical or electrochemical properties of molecules in close proximity to them in response to a change in the conformation or the activity of the nucleic acid sensor molecule following recognition of a target molecule by the target modulation domain.

As defined herein, “specificity” refers to the ability of a nucleic acid of the present invention to recognize and discriminate among competing or closely-related targets or ligands. The degree of specificity of a given nucleic acid is not necessarily limited to, or directly correlated with, the binding affinity of a given molecule. For example, hydrophobic interaction between molecule A and molecule B has a high binding affinity, but a low degree of specificity. A nucleic acid that is 100 times more specific for target A relative to target B will preferentially recognize and discriminate for target A 100 times better than it recognizes and discriminates for target B.

As defined herein, “selective” refers to a molecule that has a high degree of specificity for a target molecule.

The invention is based in part on the discovery of compositions that include a nucleic acid moiety linked to a cytotoxic agent. The nucleic acid moiety binds to a desired cell or cell surface marker. The linked cytotoxic agent is thus brought in close proximity of the cell, which allows for the cytotoxic agent to exert its cytotoxic effects on the cell. The use of these aptamer-toxin conjugates allows for the selective delivery of cytotoxic molecules to target cells.

In one aspect, the invention provides an aptamer-toxin conjugate wherein the toxin is a chemotoxin. In some embodiments, the toxin is a protein toxin. In other embodiments, the toxin is a nucleic acid toxin.

In some embodiments, the toxin is attached to the aptamer through covalent bond. If desired, the toxin is attached to an aptamer through a hydrolysable bond, and/or through a bond that can be cleaved through enzymatic activity.

In other embodiments, the toxin is attached to the aptamer through a non-covalent bond.

In some embodiments, the aptamer-toxin conjugate binds to target, thereby delivering toxin to the vicinity of the target. The toxin may interact with the same target, or with a second target in the vicinity of the first target.

In some embodiments, binding to the target results in the translocation of the aptamer and associated toxin. For example, binding to the target results in the translocation of the aptamer and associated toxin across a cell membrane. In some embodiments, binding to target results in the translocation of the aptamer and associated toxin through structures in an organ, tissue or cell.

In some embodiments, the aptamer-toxin conjugate binds to a target, and binding to target results in a change in conformation of the aptamer-toxin. The change in conformation results in a change in activity of the aptamer-toxin.

For example, in some embodiments, binding of the aptamer-toxin conjugate to a target can result in a change in conformation of the aptamer-toxin conjugate, such change resulting in a release of the toxin.

Alternatively, or in addition, binding of the aptamer-toxin conjugate to a target can result in a change in conformation of the aptamer-toxin conjugate, wherein the conformational change results in an activation of the toxin.

In a further embodiment, the aptamer-toxin conjugate binds to a target, where binding to target results in a change in conformation of the aptamer-toxin conjugate, and the change results in inactivation of the toxin.

In various embodiments, an aptamer-toxin conjugate is provided whose half-life is less than, equal to, or greater than, the half-life of the toxin.

Also provided by the invention is a method of generating an aptamer-toxin conjugate that includes attaching a toxin to an aptamer. In some embodiments, the aptamer in the moiety is created using a process termed “Systematic Evolution of Ligands by EXponential enrichment” (the “SELEX process”). The SELEX process is a method for the in vitro evolution of nucleic acid molecules with highly specific binding to target molecules and is described in, e.g., U.S. Pat. No. 5,475,096 entitled “Nucleic Acid Ligands”, and U.S. Pat. No. 5,270,163 (see also WO91/19813) entitled “Nucleic Acid Ligands”.

For example, the invention includes a method of generating an aptamer-toxin conjugate by attaching a toxin to a random pool of nucleic acids and then using the SELEX process to find the optimized aptamer-toxin conjugate from within the random pool. Alternatively, a toxin can be attached to an aptamer post-selection.

In some embodiments, the method of generating an aptamer-toxin conjugate results in a aptamer whose half-life is engineered to match the half life of the toxin. For example, the invention includes a method of generating an aptamer-toxin conjugate where the aptamer half life is engineered to match the half life of the toxin by adjusting the percentage of nuclease resistant bases in the aptamer. In other embodiments, the invention includes a method of generating an aptamer-toxin conjugate where the aptamer half life is engineered to match the half life of the toxin by changing the 5′ and/or 3′ end capping.

Also within the invention is a NASM-toxin conjugate wherein the toxin is a chemotoxin. In some embodiments, the toxin is a protein toxin. In other embodiments, the toxin is a nucleic acid toxin.

In some embodiments, the toxin is attached to the NASM through covalent bond. If desired, the toxin is attached to a NASM through a hydrolysable bond, and/or through a bond that can be cleaved through enzymatic activity.

In other embodiments, the toxin is attached to the NASM through a non-covalent bond.

In some embodiments, the NASM-toxin conjugate binds to target, thereby delivering toxin to the vicinity of the target. The toxin may interact with the same target, or with a second target in the vicinity of the first target.

In some embodiments, binding to the target results in the translocation of the NASM and associated toxin. For example, binding to the target results in the translocation of the NASM and associated toxin across a cell membrane. In some embodiments, binding to target results in the translocation of the NASM and associated toxin through structures in a organ, tissue or cell.

In some embodiments, the NASM-toxin conjugate binds to a target, and binding to target results in a change in conformation of the NASM-toxin conjugate. The change in conformation results in a change in activity of the NASM-toxin.

For example, in some embodiments, binding of the NASM-toxin conjugate to a target can result in a change in conformation of the NASM-toxin conjugate, such change resulting in a release in the toxin.

Alternatively, or in addition, binding of the NASM-toxin conjugate to a target can result in a change in conformation of the NASM-toxin conjugate, wherein the conformational change results in an activation of the toxin.

In a further embodiment, the NASM-toxin conjugate binds to a target, where binding to target results in a change in conformation of the NASM-toxin conjugate, and the change results in inactivation of the toxin.

In various embodiments, a NASM-toxin conjugate is provided whose half-life is less than, equal to, or greater than, the half-life of the toxin.

Also provided by the invention is a method of generating a NASM-toxin conjugate that includes attaching a toxin to a NASM. In some embodiments, the NASM in the moiety is created using a process similar to the SELEX process described above. However, rather than select for molecules with increased binding affinities, molecules are selected on the basis of their catalytic ability, i.e., their ability to turn the NASM on or off.

For example, the invention includes a method of generating a NASM-toxin conjugate by attaching a toxin to an a random pool of nucleic acids and then using the SELEX-like process described above to find the optimized NASM-toxin conjugate from within the random pool.

In some embodiments, the method of generating a NASM-toxin conjugate results in a NASM whose half-life is engineered to match the half life of the toxin. For example, the invention includes a method of generating a NASM-toxin conjugate where the NASM half life is engineered to match the half life of the toxin by adjusting the percentage of nuclease resistant bases in the NASM. In other embodiments, the invention includes a method of generating a NASM-toxin conjugate where the NASM half life is engineered to match the half life of the toxin by changing the 5′ and/or 3′ end capping.

The aptamer-toxins and/or NASM-toxins can be engineered so that the nucleic acid moiety recognizes a transporter, e.g., a folate transporter or an amino acid transporter (including a valine, arginine, lysine, or histidine transporter), a peptide transporter, a nucleotide transporter, or a sugar or carbohydrate transporter. Alternatively, or in addition, the nucleic acid moiety can be engineered to recognize a receptor that is internalized upon ligand binding, e.g., a receptor such as Her 2, EGF, glucose.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Nucleic Acid Compositions

In addition to carrying genetic information, nucleic acids can adopt complex three-dimensional structures. These three-dimensional structures are capable of specific recognition of target molecules and, furthermore, of catalyzing chemical reactions. Nucleic acids will thus provide candidate detection molecules for diverse target molecules, including those which do not naturally recognize or bind to DNA or RNA.

In aptamer selection, combinatorial libraries of oligonucleotides are screened in vitro to identify oligonucleotides which bind with high affinity to pre-selected targets. In NASM selection, on the other hand, combinational libraries of oligonucleotides are screened in vitro to identify oligonucleotides which exhibit increased catalytic activity in the presence of targets. Possible target molecules for both aptamers and NASMS include natural and synthetic polymers, including proteins, polysaccharides, glycoproteins, hormones, receptors, and cell surfaces, and small molecules such as drugs, metabolites, transition state analogs, specific phosphorylation states, and toxins. Small biomolecules, e.g., amino acids, nucleotides, NAD, S-adenosyl methionine, chloramphenicol, and large biomolecules, e.g., thrombin, Ku, DNA polymerases, are effective targets for aptamers, catalytic RNAs (ribozymes) discussed herein (e.g., hammerhead RNAs, hairpin RNAs) as well as NASMs.

While the aptamer selection processes described identifies aptamers through affinity-based (binding) selections, the selection processes as described for NASMs identifies nucleic acid sensor molecules through target modulation of the catalytic core of a ribozyme. In NASM selection, selective pressure on the starting population of NASMs (starting pool size is as high as 10¹⁴ to 10¹⁷ molecules) results in nucleic acid sensor molecules with enhanced catalytic properties, but not necessarily in enhanced binding properties. Specifically, the NASM selection procedures place selective pressure on catalytic effectiveness of potential NASMS by modulating both target concentration and reaction time-dependence. Either parameter, when optimized throughout the selection, can lead to nucleic acid molecular sensor molecules which have custom-designed catalytic properties, e.g., NASMs that have high switch factors, and or NASMs that have high specificity.

Aptamers

Systematic Evolution of Ligands by Exponential Enrichment, “SELEX™,” is a method for making a nucleic acid ligand for any desired target, as described, e.g., in U.S. Pat. Nos. 5,475,096; 5,670,637; 5,696,249; 5,270,163; 5,707,796; 5,595,877; 5,660,985; 5,567,588; 5,683,867; 5,637,459; 5,705,337; 6,011,020; 5,789,157; 6,261,774; EP 0 553 838 and PCT/US91/04078, each of which is specifically incorporated herein by reference.

SELEX™ technology is based on the fact that nucleic acids have sufficient capacity for forming a variety of two- and three-dimensional structures and sufficient chemical versatility available within their monomers to act as ligands (i.e., form specific binding pairs) with virtually any chemical compound, whether large or small in size.

The method involves selection from a mixture of candidates and step-wise iterations of structural improvement, using the same general selection theme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired.

Within a nucleic acid mixture containing a large number of possible sequences and structures, there is a wide range of binding affinities for a given target. A nucleic acid mixture comprising, for example a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20-50 nucleotides.

The basic SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes a SELEX™ based method for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target. Each of these patents and applications is specifically incorporated herein by reference.

SELEX™ can also be used to obtain nucleic acid ligands that bind to more than one site on the target molecule, and to nucleic acid ligands that include non-nucleic acid species that bind to specific sites on the target.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

The random sequence portion of the oligonucleotide is flanked by at least one fixed sequence which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores (described further below), sites for selective binding to affinity columns, and other sequences to facilitate cloning and/or sequencing of an oligonucleotide of interest.

In one embodiment, the random sequence portion of the oligonucleotide is about 15-70 (e.g., about 30-40) nucleotides in length and can comprise ribonucleotides and/or deoxyribonucleotides. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The SELEX™ method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX™-identified nucleic acid ligands containing modified nucleotides are described in U.S. Pat. No. 5,660,985, which describes oligonucleotides containing nucleotide derivatives chemically modified at the 5′ and 2′ positions of pyrimidines. U.S. Pat. No. 5,756,703 describes oligonucleotides containing various 2′-modified pyrimidines. U.S. Pat. No. 5,580,737 describes highly specific nucleic acid ligands containing one or more nucleotides modified with 2′-amino (2′-NH₂), 2′-fluoro (2′-F), and/or 2′-O-methyl (2′-OMe) substituents.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described in U.S. Pat. No. 6,011,020.

SELEX™ identified nucleic acid ligands that are associated with a lipophilic compound, such as diacyl glycerol or dialkyl glycerol, in a diagnostic or therapeutic complex are described in U.S. Pat. No. 5,859,228. Nucleic acid ligands that are associated with a lipophilic compound, such as a glycerol lipid, or a non-immunogenic high molecular weight compound, such as polyalkylene glycol are further described in U.S. Pat. No. 6,051,698. See also PCT Publication No. WO 98/18480. These patents and applications allow the combination of a broad array of shapes and other properties, and the efficient amplification and replication properties, of oligonucleotides with the desirable properties of other molecules.

The identification of nucleic acid ligands to small, flexible peptides via the SELEX™ method has been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). The use of 2-fluoro-ribonucleotide oligomer molecules can increase the sensitivity of an aptamer for a target molecule by ten-to-one hundred-fold over those generated using unsubstituted ribo- or deoxyribooligonucleotides (Pagratis, et al., Nat. Biotechnol. 15:6873 (1997)), providing additional binding interactions with a target molecule and increasing the stability of the secondary structure(s) of the aptamer (Kraus, et al., Journal of Immunology 160:5209-5212 (1998); Pieken, et al., Science 253:314-317 (1991); Lin, et al., Nucl. Acids Res. 22:5529-5234 (1994); Jellinek, et al. Biochemistry 34:11363-11372 (1995); Pagratis, et al., Nat. Biotechnol 15:68-73 (1997)).

Nucleic acid aptamer molecules are generally selected in a 5 to 20 cycle procedure. In one embodiment, heterogeneity is introduced only in the initial selection stages and does not occur throughout the replicating process.

The starting library of DNA sequences is generated by automated chemical synthesis on a DNA synthesizer. This library of sequences is transcribed in vitro into RNA using T7 RNA polymerase and purified. In one example, the 5′-fixed:random:3′-fixed sequence is separated by a random sequence having 30 to 50 nucleotides. Alternatively, the starting library can also be random RNA sequences synthesized on an RNA synthesizer.

Sorting among the billions of aptamer candidates to find the desired molecules starts from the complex sequence pool, whereby desired aptamers are isolated through an iterative in vitro selection process. The selection process removes both non-specific and non-binding types of contaminants. In a following amplification stage, thousands of copies of the surviving sequences are generated to enable the next round of selection. During amplification, random mutations can be introduced into the copied molecules—this ‘genetic noise’ allows functional nucleic acid aptamer molecules to continuously evolve and become even better adapted. The entire experiment reduces the pool complexity from 10¹⁷ molecules down to around 100 aptamer candidates that require detailed characterization.

Aptamer selection is accomplished by passing a solution of oligonucleotides through a column containing the target molecule. The flow-through, containing molecules which are incapable of binding target, is discarded. The column is washed, and the wash solution is discarded. Oligonucleotides which bound to the column are then specifically eluted, reverse transcribed, amplified by PCR (or other suitable amplification techniques), transcribed into RNA, and then reapplied to the selection column. Successive rounds of column application are performed until a pool of aptamers enriched in target binders is obtained.

Negative selection steps can also be performed during the selection process. Addition of such selection steps is useful to remove aptamers which bind to a target in addition to the desired target. Additionally, where the target column is known to contain an impurity, negative selection steps can be performed to remove from the binding pool those aptamers which bind selectively to the impurity, or to both the impurity and the desired target. For example, where the desired target is known, care must be taken so as to remove aptamers which bind to closely related molecules or analogs. Examples of negative selection steps include, for example, incorporating column washing steps with analogs in the buffer, or the addition of an analog column before the target selection column (e.g., the flow through from the analog column will contain aptamers which do not bind the analog).

After the completion of selection, the target-specific aptamers are reverse transcribed into DNA, cloned and amplified.

Aptamers can additionally include aptamer beacons as described, e.g., WO 00/70329. The publication discloses compositions, systems, and methods for simultaneously detecting the presence and quantity of one or more different compounds in a sample using aptamer beacons. Aptamer beacons are oligonucleotides that have a binding region that can bind to a non-nucleotide target molecule, such as a protein, a steroid, or an inorganic molecule. New aptamer beacons having binding regions configured to bind to different target molecules can be used in solution-based and solid, array-based systems. The aptamer beacons can be attached to solid supports, e.g., at different predetermined points in two-dimensional arrays.

Nucleic Acid Sensor Molecules (NASMs)

Nucleic acid sensor molecules are nucleic acid molecules (e.g., DNA or RNA molecules) that include a target recognition domain, a catalytic domain, and, optionally, a linker domain connecting the catalytic domain. Thus, NASMs include allosteric ribozymes, whose activity is switched on or off by the presence of a specific target. Allosteric ribozymes can act as reporter molecules in that they directly couple molecular detection to the triggering of a chemical reaction. Because they are also target molecule specific, however, they can also be used in much the same way as aptamers, e.g., to deliver toxins to a target. The combination of these properties in a single molecule makes them powerful tools for a wide range of applications.

Nucleic acid sensor molecules suitable for use in the compositions and methods of the invention are disclosed in, e.g., WO 03/014375 which is incorporated herein by reference.

Nucleic acid-based detection schemes have exploited the ligand-sensitive catalytic properties of some nucleic acids, e.g., such as ribozymes. Ribozyme-based nucleic acid sensor molecules have been designed both by engineering and by in vitro selection methods. Some engineering methods exploit the apparently modular nature of nucleic acid structures by coupling molecular recognition to signaling by simply joining individual target-modulation and catalytic domains using, e.g., a double-stranded or partially double-stranded linker. ATP sensors, for example, have been created by appending the previously-selected, ATP-selective sequences (see, e.g., Sassanfar et al., Nature 363:550-553 (1993)) to either the self-cleaving hammerhead ribozyme (see, e.g., Tang et al., Chem. Biol. 4:453-459 (1997)) as a hammerhead-derived sensor, or the L1 self-ligating ribozyme (see, e.g., Robertson et al., Nucleic Acids Res. 28:1751-1759 (2000)) as a ligase-derived sensor. Hairpin-derived sensors are also contemplated. In general, the target modulation domain is defined by the minimum number of nucleotides sufficient to create a three-dimensional structure which recognizes a target molecule.

Catalytic nucleic acid sensor molecules (NASMs) are selected which have a target molecule-sensitive catalytic activity (e.g., self-cleavage) from a pool of randomized or partially randomized oligonucleotides. The catalytic NASMs have a target modulation domain which recognizes the target molecule and a catalytic domain for mediating a catalytic reaction induced by the target modulation domain's recognition of the target molecule. Recognition of a target molecule by the target modulation domain triggers a conformational change and/or change in catalytic activity in the nucleic acid sensor molecule. In one embodiment, by modifying (e.g., removing) at least a portion of the catalytic domain and coupling it to an optical signal generating unit, an optical nucleic acid sensor molecule is generated whose optical properties change upon recognition of the target molecule by the target modulation domain. In one embodiment, the pool of randomized oligonucleotides comprises the catalytic site of a ribozyme.

A heterogeneous population of oligonucleotide molecules comprising randomized sequences is screened to identify a nucleic acid sensor molecule having a catalytic activity which is modified (e.g., activated) upon interaction with a target molecule. As with the aptamer nucleic acids, the oligonucleotide can be RNA, DNA, or mixed RNA/DNA, and can include modified or nonnatural nucleotides or nucleotide analogs.

Each oligonucleotide in the population comprises a random sequence and at least one fixed sequence at its 5′ and/or 3′ end. In one embodiment, the population comprises oligonucleotides which include as fixed sequences an aptamer known to specifically bind a particular target and a catalytic ribozyme or the catalytic site of a ribozyme, linked by a randomized oligonucleotide sequence. In a preferred embodiment, the fixed sequence comprises at least a portion of a catalytic site of an oligonucleotide molecule (e.g., a ribozyme) capable of catalyzing a chemical reaction.

Catalytic sites are well known in the art and include, e.g., the catalytic core of a hammerhead ribozyme (see, e.g., U.S. Pat. No. 5,767,263; U.S. Pat. No. 5,700,923) or a hairpin ribozyme (see, e.g., U.S. Pat. No. 5,631,359). Other catalytic sites are disclosed in U.S. Pat. No. 6,063,566; Koizumi et al., FEBS Lett. 239: 285-288 (1988); Haseloff and Gerlach, Nature 334: 585-59 (1988); Hampel and Tritz, Biochemistry 28: 49294933 (1989); Uhlenbeck, Nature 328: 596-600 (1987); and Fedor and Uhlenbeck, Proc. Natl. Acad. Sci. USA 87: 1668-1672 (1990).

In some embodiments, a population of partially randomized oligonucleotides is generated from known aptamer and ribozyme sequences joined by the randomized oligonucleotides. Most molecules in this pool are non-functional, but a handful will respond to a given target and be useful as nucleic acid sensor molecules. Catalytic NASMs are isolated by the iterative process described above. In all embodiments, during amplification, random mutations can be introduced into the copied molecules—this ‘genetic noise’ allows functional NASMs to continuously evolve and become even better adapted as target-activated molecules.

In another embodiment, the population comprises oligonucleotides which include a randomized oligonucleotide linked to a fixed sequence which is a catalytic ribozyme, the catalytic site of a ribozyme or at least a portion of a catalytic site of an oligonucleotide molecule (e.g., a ribozyme) capable of catalyzing a chemical reaction. The starting population of oligonucleotides is then screened in multiple rounds (or cycles) of selection for those molecules exhibiting catalytic activity or enhanced catalytic activity upon recognition of the target molecule as compared to the activity in the presence of other molecules, or in the absence of the target.

The nucleic acid sensor molecules identified through in vitro selection, e.g., as described above, comprise a catalytic domain (i.e., a signal generating moiety), coupled to a target modulation domain, (i.e., a domain which recognizes a target molecule and which transduces that molecular recognition event into the generation of a detectable signal). In addition, the nucleic acid sensor molecules of the present invention use the energy of molecular recognition to modulate the catalytic or conformational properties of the nucleic acid sensor molecule.

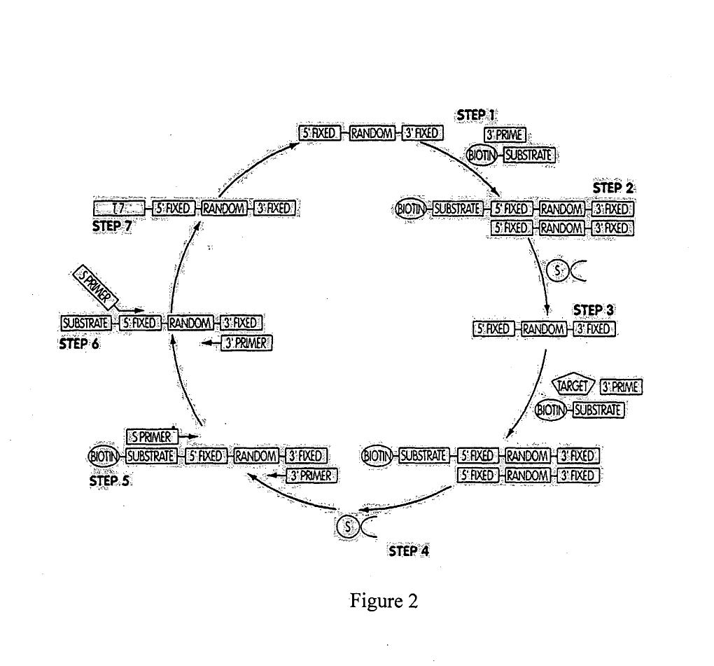

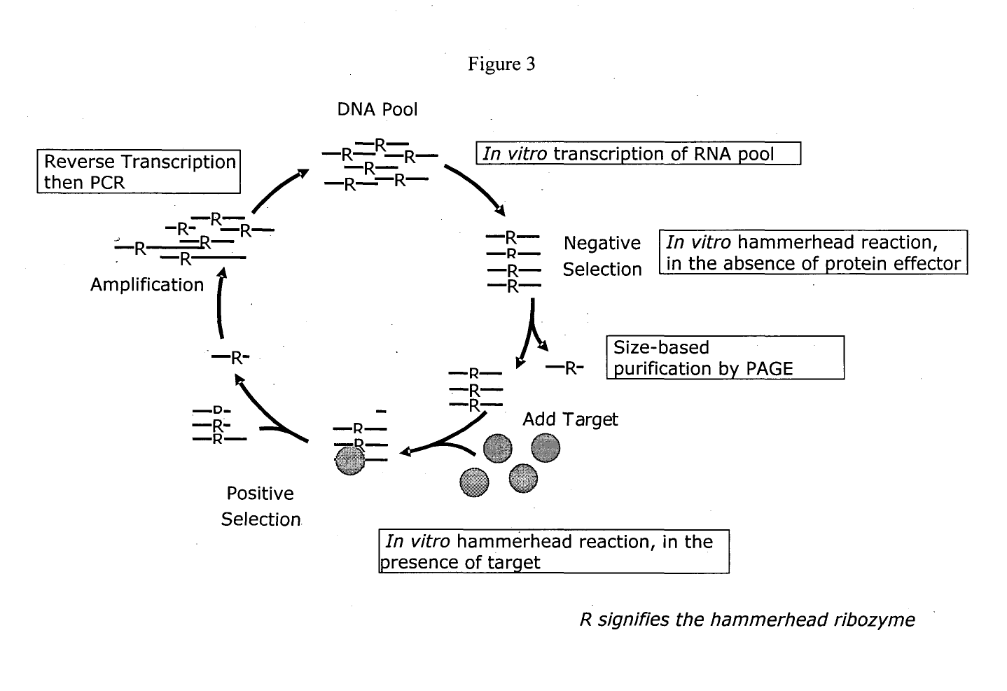

Nucleic acid sensor molecules are generally selected in a 5 to 20 cycle procedure. In one embodiment, heterogeneity is introduced only in the initial selection stages and does not occur throughout the replicating process. FIG. 2 shows a schematic diagram in which the oligonucleotide population is screened for a nucleic acid sensor molecule which comprises a target molecule activatable ligase activity. FIG. 3 shows the hammerhead nucleic acid sensor molecule selection methodology. Each of these methods are readily modified for the selection of NASMs with other catalytic activities.

Additional procedures may be incorporated in the various selection schemes, including: pre-screening, negative selection, etc. For example, individual clones isolated from selection experiments are tested early for allosteric activation in the presence of target-depleted extracts as a pre-screen, and molecules that respond to endogenous non-specific activators are eliminated from further consideration as target-modulated NASMs; to the extent that all isolated NASMs are activated by target-depleted extracts, depleted extracts are included in a negative selection step of the selection process; commercially available RNase inhibitors and competing RNAse substrates (e.g., tRNA) may be added to test samples to inhibit nucleases; or by carrying out selection in the presence of nucleases (e.g., by including depleted extracts during a negative selection step) the experiment intrinsically favors those molecules that are resistant to degradation; covalent modifications to RNA that can render it highly nuclease-resistant can be performed (e.g., 2′-O-methylation) to minimize non-specific cleavage in the presence of biological samples (see, e.g., Usman et al.). Clin. Invest. 106:1197-202 (2000).

In one embodiment, nucleic acid sensor molecules are selected which are activated by target molecules comprising molecules having an identified biological activity (e.g., a known enzymatic activity, receptor activity, or a known structural role); however, in another embodiment, the biological activity of at least one of the target molecules is unknown (e.g., the target molecule is a polypeptide expressed from the open reading frame of an EST sequence, or is an uncharacterized polypeptide synthesized based on a predicted open reading frame, or is a purified or semi-purified protein whose function is unknown).

Although in one embodiment the target molecule does not naturally bind to nucleic acids, in another embodiment, the target molecule does bind in a sequence specific or nonspecific manner to a nucleic acid ligand. In a further embodiment, a plurality of target molecules binds to the nucleic acid sensor molecule. Selection for NASMs specifically responsive to a plurality of target molecules (i.e., not activated by single targets within the plurality) may be achieved by including at least two negative selection steps in which subsets of the target molecules are provided. Nucleic acid sensor molecules can be selected which bind specifically to a modified target molecule but which do not bind to closely related target molecules. Stereochemically distinct species of a molecules can also be targeted.

Toxins

Toxins useful in the present invention include chemotoxins having cytotoxic effects. These can be classified in their mode of action: 1) tubulin stabilizers/destabilizers; 2) anti-metabolites; 3) purine synthesis inhibitors; 4) nucleoside analogs; and 5) DNA alkylating or modifying agents. Radioisotopes also have cytotoxic effects useful in the present invention.

Examples of suitable toxins include, e.g., chemotherapeutic agents. Chemotherapeutics are typically small chemical entities produced by chemical synthesis and include cytotoxic drugs, cytostatic drugs as well as compounds which affect cells in other ways such as reversal of the transformed state to a differentiated state or those which inhibit cell replication. Examples of chemotherapeutics include, but are not limited to: methotrexate (amethopterin), doxorubicin (adrimycin), daunorubicin, cytosinarabinoside, etoposide, 5-4 fluorouracil, melphalan, chlorambucil, and other nitrogen mustards (e.g., cyclophosphamide), cis-platinum, vindesine (and other vinca alkaloids), mitomycin and bleomycin.

Toxins can include complex toxic products of various organisms including bacteria, plants, etc. Examples of toxins include but are not limited to: ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin. Protein toxins may be produced using recombinant DNA techniques as fusion proteins which include peptides of the invention. Protein toxins may also be conjugated to compounds of the invention by non-peptidyl bonds. In addition, photosensitizers and cytokines can also be used with the present invention.

Cytotoxic molecules that can be used in the present invention are anthracycline family of cytotoxic agents, e.g., doxorubicin (DOX). Doxorubicin damages DNA by intercalation of anthracycline protion, metal ion, chelation, or by generation of free radicals. DOX has also been shown to inhibit DNA topoisomerase II. Doxorubicin has been shown clinically to have broad spectrum of activity and toxic side effects that are both dose-related and predictable. Efficacy of DOX is limited by myelosuppression and cardiotoxicity. Complexed with a targeting moiety such as an aptamer increases intratumoral accumulation while reducing systemic exposure.

Maytansinoids are very toxic chemotherapeutic molecules that can be used as therapeutic moieties of the present invention. Maytansinoids effect their cytotoxicity by inhibiting tubulin polymerization, thus inhibiting cell division and proliferation. Maytansinoid derivative DM1 has been conjugated to other targeting moieties, e.g., murine IgG1 mAb against MUC-1 and to an internalizing anti-PSMA murine monoclonal antibody 8D11 (mAb) through disulfide linker chemistry.

Enediynes are another class of cytotoxic molecules that can be used as therapeutic moieties of the present invention. Enediynes effect their cytotoxicity by producing double-stranded DNA breaks at very low drug concentrations. The enediynes class of compounds includes calicheamicins, neocarzinostatin, esperamicins, dynemicins, kedarcidin, and maduropeptin. Linking chemistries for these compounds include periodate oxidation of carbohydrate residues followed by reaction with a hydrazide derivative of calicheamycin, for example. These conjugates utilize an acid-labile hydrazone bond to a targeting moiety, such as a monoclonal antibody to ensure hydrolysis following internalization into lysosomes, and a sterically protected disulfide bond to calicheamicin to increase stability in circulation.

Tumor therapeutics also include radionuclides, particularly high energy alpha particle emitters. Alpha particles are high energy, high linear energy transfer (LET) helium nuclei capable of strong, yet selective cytotoxicity. Approximately 100 radionuclides decay with alpha emission. A single atom emitting an alpha particle can have a lethal cytotoxic effect on a single cell. Conjugates of radionuclides to mAbs have been used in preclinical models of leukemia and prostate cancer, and a phase I clinical trial is underway with ²¹¹At-labeled anti tenascin mAb against malignant gliomas.

Radioisotopes may be conjugated to compounds of the invention. Examples of radioisotopes which are useful in radiation therapy include, e.g. ⁴⁷Sc, ⁶⁷Cu, ⁹⁰Y, ¹⁰⁹Pd, ¹²³I, ¹²⁵I, ¹³¹I, ¹⁸⁶Re, ¹⁸⁸Re, ¹⁹⁹Au, ²¹¹At, ²¹²Pb, ²¹²Bi. Some alpha particle emitting radioisotopes exhibit too short a half life to be effective therapeutics against most tumors. For example, ²¹³Bi has a 46 minute half life which limits its efficacy to only the most accessible cancer cells, and poses practical obstacles such as timely shipment and administration. Another radioisotope ²²⁵Ac is a more suitable radiotherapeutic because each ²²⁵Ac atom decays into several daughter atoms, four of which also emits alpha particles.

Attachment of Nucleic Acids (Aptamers and/or NASMs) to Toxins

The present invention provides materials and methods to produce bifunctional molecules that consist of a targeting moiety that localizes to target cells, e.g., tumor cells, or neovasculature, said targeting molecule coupled with a therapeutic moiety that effects a cytotoxic effect on the target cells. The present invention provides nucleic acid targeting moieties and therapeutic agents, for example cytotoxic agents (small organic molecules), radionuclides, plant and bacterial toxins, enzymes, photosensitizers, and cytokines.

Nucleic acid targeting moieties of the present invention can be attached to therapeutic moieties, e.g., toxins, using methods known in the art. For example, methods for generating blended nucleic acid ligands comprised of functional unit(s) added to provide a nucleic acid ligand with additional functions are described in U.S. Pat. No. 5,683,867, U.S. Pat. No. 6,083,696, and U.S. Pat. No. 5,705,337. The latter patent discloses methods for identifying nucleic acid ligands capable of covalently interacting with targets of interest. The nucleic acids can be associated with various functional units. The method also allows for the identification of nucleic acids that have facilitating activities as measured by their ability to facilitate formation of a covalent bond between the nucleic acid, including its associated functional unit, and its target.

Cytotoxics—Small Organic Molecule Linking Chemistries

To link nucleic acid aptamers of the present invention to small molecule cytotoxic agents that contain carboxylate groups, the latter are converted into an amine-reactive probe (e.g. NHS ester) by conventional synthetic organic reactions, and then coupled to an amine oligonucleotide aptamer. Amine-containing small molecules can be coupled to an activated oligo (e.g. 5′-carboxy-modifier C10 (Glen Research) according to the Glen technical product bulletin). Alternatively, an amine-oligo can be activated in situ by crosslinking reagents, including but not limited to DSS, BS³ or related reagents (Pierce, Rockford, Ill.), and further coupled to amines.

Thiol-containing small molecules can be coupled to 2,2-dithio-bispyridine activated thiol aptamer or an SPDP-activated (Pierce, Rockford, Ill.) amine-oligo.

Small molecules that do not contain carboxylate, amine or thiol groups are preferably converted into such by conventional synthetic organic chemistry by methods known to those of skill in the art.

Additionally, encapsulated (e.g. in liposomes) cytotoxics can also be linked to aptamers or NASMs of the present invention with acid-labile linkers, enzyme cleavable linkers used in the art for linking liposome to reactive moieties, such as activated oligonucleotides.

Acid-labile linkers include for illustration but not limitation, cis-aconityl linkers used to link anthracyclines, doxorubicin (DOX) or daunorubicin (DNR), to immunoconjugates such as several mAbs (e.g., anti-melanoma mAb 9.927); leading to released cytotoxic agents in the environment of lysozomes.

Hydrazone linkers have been used to conjugate small molecule cytotoxic agents including DNR, morpholino-DOX to anti-αvβ3 mAb LM609, and anti-Le^{y} mAb BR96. These hydrazone linkers are acid labile at pH 4.5. Other acid-sensitive anthracycline conjugates have been obtained through modification of the C-13 carbonyl group to give acylhydrazone, semicarbazones, thiosemicarbazones and oximes.

Cytotoxics—Peptides (Synthetic) Linking Chemistries

In the case of peptide cytotoxic agents, methods for coupling of synthetic peptides include synthesis of an amine-reactive activated ester (e.g., NHS) of the peptide, coupling to amine-oligo.

Another method of linking peptide cytotoxic moieties to the targeting moieties of the present invention also include synthesis of a cytotoxic peptide moiety with an extra C- or N-terminal cysteine. This can be activated with 2,2-dithio-bispyridine and coupled to a thiol-modified aptamer oligo (standard automated synthesis, final coupling with an thiol-modifier [Glen Research, Sterling, Va.]). Alternatively, the thiol-modified aptamer is activated with 2,2-dithio-bispyridine and coupled to the cys-peptide. Lastly, an amino-terminated oligo can be activated with SPDP (Pierce, Rockford, Ill.) and coupled to the cys-containing peptide. All three methods generate the conjugate coupled through a disulfide bond.

Another method of linking peptide cytotoxic moieties to the targeting moieties of the present invention also includes modification of a targeting moiety consisting of an amine-oligo with a maleimide reagent, e.g., GMBS, (Pierce, Rockford, Ill.), subsequent coupling to cyspeptide.

Another method of linking peptide cytotoxic moieties to the targeting moieties of the present invention also includes synthesis of a targeting moiety consisting of an oligo modified with 5′-carboxy-modifier C10 (Glen Research) and in-situ coupling to an amine-containing molecule (i.e. peptide) according to methods known in the art.

Another method of linking peptide cytotoxic moieties to the targeting moieties of the present invention also includes oxidizing 3′-ribo-terminated oligos with sodium meta-periodate and the resulting aldehyde reacted with amine peptides in the presence of reducing agents. In addition, C-terminal peptide hydrazides can couple to an oxidized RNA even without the aid of reducing agents.

Cytotoxics—Protein Linking Chemistries

Methods of linking cytotoxic protein moieties of the present invention to targeting moieties of the present invention are principally the same as those methods used for linking peptides.

Methods of linking protein cytotoxic protein moieties of the present invention include activation of the targeting moiety of the invention consisting of an amino-terminated oligo with e.g. SPDP or GMBS (Pierce, Rockford, Ill.), or of an thiol-oligo with 2,2-dithio-bispyridine and coupling to the cys-containing protein.

Another method of linking cytotoxic protein moieties of the invention with targeting moieties of the present invention include coupling of protein amines to an amine-containing oligo using crosslinking reagents, e.g., DSS, BS³ or related reagents (Pierce, Rockford, Ill.).

Radioisotopes Cytotoxic Moieties Linking Chemistries

Methods of linking cytotoxic moieties of the present invention consisting of radioactive metal ions (e.g., isotopes of Tc, Y, Bi, Ac, Cu etc.) to targeting moieties of the present invention include chelation with a suitable ligand, such as DOTA (Lewis, et al., Bioconjugate Chemistry 2002, 13, 1178). A generic labeling scheme would start with the synthesis of a 5′-amino-modified aptamer oligo (standard automated synthesis, final coupling with an amino-modifier [Glen Research, Sterling, Va.]). Then, the chelator is converted into an amine-reactive activated ester, and subsequently coupled to the oligo similar to the method described in Lewis, et al.

Another method of linking radionuclide cytotoxic moieties of the present invention to targeting moieties of the present invention include oxidizing 3′-ribo-terminated oligos with sodium meta-periodate and the resulting aldehyde reacted with amine-containing chelators or radiolabels in the presence of reducing agents. Alternatively, hydrazine, hydrazide, semicarbazide and thiosemicarbazide derivatives of chelators or radiolabels can be used.

Additional methods for attaching nucleic acids to non-nucleic acid molecules are disclosed in, e.g., WO 00/70329. The publication discloses compositions, systems, and methods for simultaneously detecting the presence and quantity of one or more different compounds in a sample using aptamer beacons. Aptamer beacons are oligonucleotides that have a binding region that can bind to a non-nucleotide target molecule, such as a protein, a steroid, or an inorganic molecule. New aptamer beacons having binding regions configured to bind to different target molecules can be used in solution-based and solid, array-based systems. The aptamer beacons can be attached to solid supports, e.g., at different predetermined points in two-dimensional arrays.

Pharmaceutical Compositions

The invention also includes pharmaceutical compositions containing aptamer-toxin molecules. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

In practice, the compounds or their pharmaceutically acceptable salts, are administered in amounts which will be sufficient to induce lysis of a desired cell.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixers, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer-toxin and/or NASM molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and crosslinked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 1000 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Effective plasma levels of the compounds of the present invention range from 0.002 mg to 50 mg per kg of body weight per day.

Compounds of the present invention may be administered in a single daily dose, or the total daily dosage may be administered in divided doses of two, three or four times daily.

The foregoing being a detailed description of the present invention, persons of skill in the art will understand the following examples to be illustrative of embodiments of aspects of the present invention. Persons of skill in the art will also understand that the foregoing examples are for illustration of the present invention and not limitation thereof. Accordingly, the invention is to be defined not by the preceding illustrative description but instead by the spirit and scope of the claims that follow.

### EXAMPLE 1

### PDGF Aptamer—⁹⁰Y Conjugate

A patient is identified exhibiting symptoms of a disease wherein platelet derived growth factor (PDGF) is a marker or is implicated in pathogenesis. An aptamer specific for PDGF is generated according to the SELEX™ method and/or is identified from the prior art. Examples of such aptamers are described in U.S. Pat. No. 5,723,594 incorporated by reference herein. The aptamer is synthesized according to standard methods known to those skilled in the art including phosphoramidite synthesis methods so that an amine terminus is present on the aptamer. The amine derivatized aptamer is then conjugated to a 1,4,7,10-tetraazacyclododecane-N,N,N′,N″-tetraacetic acid (DOTA) linker reagent and the ⁹⁰Y isotope is chelated to the derivatized DOTA-aptamer complex according to Lewis, et al., Bioconjugate Chemistry, 2001, 12, 320-324.

The apatamer-⁹⁰Y conjugate is then administered to the subject or patient in a therapeutically effective amount to inhibit the disease state in the subject or patient.

### EXAMPLE 2

### PDGF Aptamer—arinA Peptide Conjugates

A patient is identified exhibiting symptoms of a disease wherein platelet derived growth factor (PDGF) is a marker or is implicated in pathogenesis. An aptamer specific for PDGF is generated according to the SELEX™ method and/or is identified from the prior art. Examples of such aptamer are described in U.S. Pat. No. 5,723,594 incorporated by reference herein. The aptamer is synthesized according to methods know to those skilled in the art including phosphoramidite synthesis. The last coupling in the oligonucleotide synthesis is done using a OPeC™ reagent phosphoramidite (Glen Research, Sterling, Va.). This is done according to the following method by Stetsenko et al., New phosphoramidite reagents for the synthesis of oligonucleotides containing a cysteine residue useful in peptide conjugation., Nucl. Acids (2000) 19, 1751-1764. The cytotoxic peptide is synthesized according to standard methods using the Pentafluorophenyl S-benzylthiosuccinate, Peptide Modifying Reagent (PMR) reagent in the final coupling step in standard Fmoc-based solid-phase peptide assembly. The conjugation of the reactive aptamer and the arinA cytotoxic peptide is done by methods described in Stetsenko, et al.

Once an aptamer-peptide conjugate has been synthesized, the therapeutic conjugate is administered to a subject or patient in a therapeutically effective amount to treat the disease state in the subject or patient. The PDGF aptamer targeting moiety brings the cytotoxic peptide in close proximity to the target cell and the peptide exerts its cytotoxic effect on the cell having a PDGF marker.

### EXAMPLE 3

### PDGF Aptamer—Protein Conjugate

A patient is identified exhibiting symptoms of a disease wherein platelet derived growth factor (PDGF) is a marker or is implicated in pathogenesis. An aptamer specific for PDGF is generated according to the SELEX method and/or is identified from the prior art. Examples of such aptamers are described in U.S. Pat. No. 5,723,594 incorporated by reference herein. The aptamer is synthesized according to methods know to those skilled in the art including phosphoramidite synthesis and so that a thiol from a cysteine reactive terminus is present in the modified aptamer to be linked. This is done according to the method by Tung, et al., Bioconjugate Chemistry, 2000, 11, 605-618. The cysteine derivatized aptamer is then conjugated to the cytotoxic protein by a peptide modifying reagent linker having a reactive group that forms a covalent bond with the —SH reactive end of the modified oligo. This results in an oligonucleotide-peptide conjugate as described by Tung, et al.

Once the therapeutic conjugate is synthesized, it is administered to a subject or patient in a therapeutically effective amount to treat the disease state in the subject or patient. The PDGF aptamer targeting moiety brings the cytotoxic protein in close proximity to the target cell and the protein exerts its cytotoxic effect on the cell having a PDGF marker.

### EXAMPLE 4

### PDGF Aptamer—DNR/DOX Chemotoxic Organic Molecule Conjugate

A patient is identified exhibiting symptoms of a disease wherein platelet derived growth factor (PDGF) is a marker or is implicated in pathogenesis. An aptamer specific for PDGF is generated according to the SELEX™ method and/or is identified from the prior art. Examples of such aptamers are described in U.S. Pat. No. 5,723,594 incorporated by reference herein. The aptamer is synthesized according to methods know to those skilled in the art including hydrazidephosphoramidite synthesis so that a carbonyl reactive terminus is present. This is done according to the following method by Raddatz, et al., Hydrazide oligonucleotides: new chemical modification for chip array attachment and conjugation. Nucleic Acids Res., Nov. 1, 2002:30(21):4793-802. The hydrazide derivatized aptamer is then conjugated to the carbonyl functional group of the DOX or DNR chemotoxic organic molecule according to Trail, et al., Cancer Immunol Immunother, (2003) 52:328-337, and references cited therein.

Once the PDGF aptamer-DOX or DNR conjugate is created it is administered to the subject or patient having a proliferative disease where PDGF is a marker and is involved in its pathogenesis. Once the DOX/DNR is brought in close proximity of the target cell by the PDGF specific aptamer, the DOX/DNR cytotoxic moiety exerts its cytotoxic effect on the targeted cells reducing non-specific collateral damage to non-target cells or surrounding tissue.

References cited are incorporated by reference herein in their entirety.

The present invention having been described by detailed description and the non-limiting examples above, is now defined by the spirit and scope of the following claims.

What is claimed is:

1) An aptamer-toxin conjugate therapeutic agent comprising a targeting moiety conjugated to a cytotoxic moiety.

2) The therapeutic agent of claim 1 wherein said targeting moiety is an aptamer.

3) The therapeutic agent of claim 1 wherein said targeting moiety is a nucleic acid sensor molecule.

4) The therapeutic agent of claim 2 wherein said cytotoxic moiety is selected from the group consisting of a cytotoxic peptide, a cytotoxic protein, a small molecule chemotherapeutic agent, and a radioisotope therapeutic molecule.

5) The therapeutic agent of claim 3 wherein said cytotoxic moiety is selected from the group consisting of a cytotoxic peptide, a cytotoxic protein, a small molecule chemotherapeutic agent, and a radioisotope therapeutic molecule.

6) The therapeutic agent of claim 4, wherein said targeting moiety is conjugated to said cytotoxic moiety by a covalent bond.

7) The therapeutic agent of claim 5, wherein said targeting moiety is conjugated to said cytotoxic moiety by a covalent bond.

8) The therapeutic agent of claim 4 wherein said targeting moiety is conjugated to said cytotoxic moiety by a non-covalent bond.

9) The therapeutic agent of claim 5 wherein said targeting moiety is conjugated to said cytotoxic moiety by a non-covalent bond.
